(19) United States
(12) Patent Application Publication
(10) Pub. No.: US 2026/0077349 A1
(43) Pub. Date: Mar. 19, 2026

(54) OPTICAL TEST STRIP SYSTEM

(71) Applicant: Fluid Photonics Corporation, Plainview, NY (US)

(72) Inventors: Dean G. Gouramanis, Lindenhurst, NY (US); Jason John Poulos, Commack, NY (US)

(73) Assignee: Fluid Photonics Corporation, Plainview, NY (US)

(21) Appl. No.: 19/239,936

(22) Filed: Jun. 16, 2025

Related U.S. Application Data
(60) Provisional application No. 63/660,525, filed on Jun. 16, 2024.

(51) Int. Cl. B01L 3/00

(52) U.S. Cl. CPC B01L 3/502715; B01L 3/502707; B01L 2200/027; B01L 2300/0825

(57) ABSTRACT

An optical test strip reader assembly is disclosed that includes a chassis, a reagent functionalization tool including a plurality of standard components installed in the chassis, a plurality of instrumentation modules that are each configured for assembly with the reagent functionalization tool in the chassis, each instrumentation module being selectable for assembly with the reagent functionalization tool in the chassis based on at least one parameter that corresponds to a target application for the optical test strip reader, and an optical test strip inlet that is configured to receive a portion of an optical test strip therein and to align a sample region of the optical test strip within the chassis at a location corresponding to a given instrumentation module of the plurality of instrumentation modules when the given instrumentation module is assembled within the chassis.

### COPYRIGHT NOTICE

A portion of the disclosure of this patent document contains material, which is subject to copyright protection. The copyright owner has no objection to the facsimile reproduction by anyone of the patent document or the patent disclosure, as it appears in the Patent and Trademark Office patent files or records, but otherwise reserves all copyright rights whatsoever.

### CROSS REFERENCE TO RELATED APPLICATION

This application claims the benefit of U.S. Provisional Ser. No. 63/660,525 , entitled “OPTICAL TEST STRIP SYSTEM,” filed on Jun. 16, 2024, the entirety of which is incorporated by reference herein.

This application is related to the following patents and applications, which are incorporated by reference herein in their entirety:
U.S. Provisional Ser. No. 68/647,618 , entitled “MICROELECTRONIC CHEMICAL CONCENTRATION SENSOR FUNCTIONALIZATION TOOL,” filed on May 15, 2024,
PCT Patent Application No. PCT/US23/74423, entitled “AUTONOMOUS MEASUREMENT SYSTEM FOR PERFORMING CONCENTRATION MEASUREMENTS IN A FLUID STREAM,” filed on Sep. 17, 2023, and
U.S. patent application Ser. No. 18/340,967, entitled “AUTONOMOUS MEASUREMENT SYSTEM FOR PERFORMING CHEMICAL CONCENTRATION MEASUREMENTS IN AN INDUSTRIAL PROCESS STREAM,” filed on Jun. 26, 2024.

### BACKGROUND

This application relates to a configurable optical test strip system, and in particular, to a configurable optical test strip system comprising an optical test strip reader, optical test strips and an optical test strip loader.

### SUMMARY

In an embodiment, an optical test strip reader assembly is disclosed. The optical test strip reader assembly comprises a chassis, a reagent functionalization tool comprising a plurality of standard components installed in the chassis, a plurality of instrumentation modules that are each configured for assembly with the reagent functionalization tool in the chassis, each instrumentation module being selectable for assembly with the reagent functionalization tool in the chassis based on at least one parameter that corresponds to a target application for the optical test strip reader and an optical test strip inlet that is configured to receive a portion of an optical test strip therein and to align a sample region of the optical test strip within the chassis at a location corresponding to a given instrumentation module of the plurality of instrumentation modules when the given instrumentation module is assembled within the chassis.

In an embodiment, a method of manufacture of an optical test strip reader is disclosed. The method comprises obtaining at least one parameter corresponding to a target application, identifying an instrumentation module to include in the optical test strip reader from a plurality of available instrumentation modules based on the obtained at least one parameter and assembling the optical test strip reader by obtaining a chassis, installing a reagent functionalization tool comprising a plurality of standard components in the chassis and installing the identified instrumentation module in the chassis. The installation of the identified instrumentation module electrically connects the identified instrumentation module with the reagent functionalization tool. The method further comprises installing a bulkhead in the chassis. The bulkhead comprises an optical test strip inlet that is configured to align a sample region of an optical test strip inserted therein with the installed instrumentation module. The method further comprises sealing the chassis.

In an embodiment, an optical test strip reader is disclosed. The optical test strip reader comprises a chassis, a reagent functionalization tool comprising a plurality of standard components installed in the chassis and a first instrumentation module installed in the chassis. The first instrumentation module has been selected from a plurality of available instrument modules based on at least one parameter corresponding to a target application. The optical test strip reader comprises a second instrumentation module installed in the chassis. The second instrumentation module has been selected from the plurality of available instrument modules based on the at least one parameter corresponding to the target application. The optical test strip reader comprises a bulkhead installed in the chassis. The bulkhead comprising an optical test strip inlet that is configured to align a sample region of an optical test strip inserted therein with the first and second instrumentation modules.

The foregoing summary is illustrative only and is not intended to be in any way limiting. These and other illustrative embodiments include, without limitation, apparatus, systems, methods and computer-readable storage media. In addition to the illustrative aspects, embodiments, and features described above, further aspects, embodiments, and features will become apparent by reference to the drawings and the following detailed description.

### BRIEF DESCRIPTION OF THE DRAWINGS

The invention is illustrated in the figures of the accompanying drawings which are meant to be exemplary and not limiting, in which like references are intended to refer to like or corresponding parts.

FIG. 1 is a block diagram of a system according to an embodiment.

FIG. 2 is an exploded view of an example optical test strip reader of the system of FIG. 1 according to an embodiment.

FIG. 3 is another exploded view of the example optical test strip reader of FIG. 2 according to an embodiment.

FIG. 4 is a perspective view of the bottom half of the example optical test strip reader of FIG. 2 prior to installation of instrumentation modules according to an embodiment.

FIG. 5 is a perspective view of the bottom half of the example optical test strip reader of FIG. 2 after installation of the instrumentation modules according to an embodiment.

FIG. 6 is a top down view of an example optical test strip of the system of FIG. 1 according to an embodiment.

FIG. 7 is a side view of the example optical test strip of the system of FIG. 6 according to an embodiment.

FIG. 8 is a side view of an example multi-layer optical test strip of the system of FIG. 1 according to an embodiment.

FIG. 9 is an exploded view of the example multi-layer optical test strip of FIG. 8 according to an embodiment.

FIG. 10 is a top down view of a middle layer of the example multi-layer optical test strip of FIG. 8 according to an embodiment.

FIG. 11 is an exploded view of an example multi-layer optical test strip of the system of FIG. 1 according to an embodiment.

FIG. 12 is a front view of an example optical test strip loader of the system of FIG. 1 according to an embodiment.

FIG. 13 is a top down view of the example optical test strip loader of FIG. 12 according to an embodiment.

### DETAILED DESCRIPTION OF THE INVENTION

Subject matter will now be described more fully hereinafter with reference to the accompanying drawings, which form a part hereof, and which show, by way of illustration, exemplary embodiments in which the invention may be practiced. Subject matter may, however, be embodied in a variety of different forms and, therefore, covered or claimed subject matter is intended to be construed as not being limited to any example embodiments set forth herein; example embodiments are provided merely to be illustrative. It is to be understood that other embodiments may be utilized and structural changes may be made without departing from the scope of the illustrative embodiments. Likewise, a reasonably broad scope for claimed or covered subject matter is intended. Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of exemplary embodiments in whole or in part. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

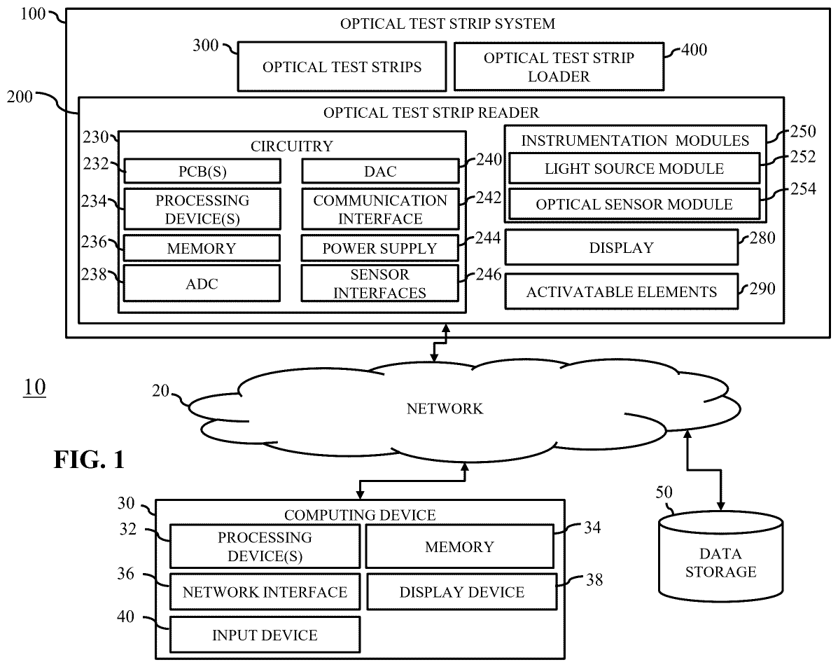

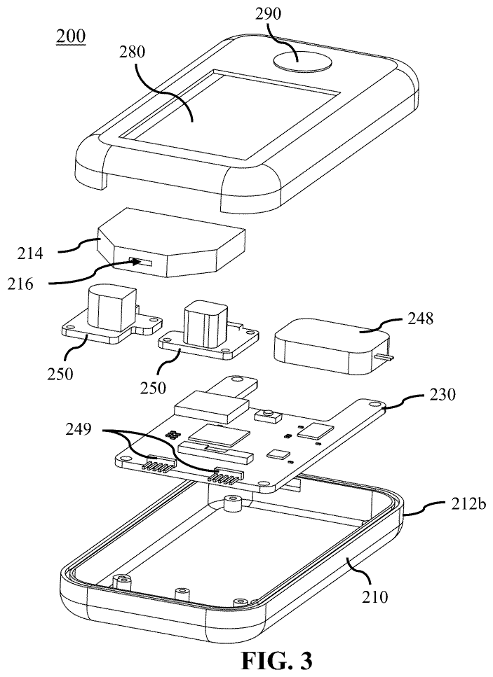

With reference to FIGS. 1-13, a system 10 is disclosed that comprises a network 20, a computing device 30, data storage 50 and an optical test strip system 100. Optical test strip system 100 comprises an optical test strip reader 200, optical test strips 300 and an optical test strip loader 400.

Network 20 is configured to connect optical test strip reader 200, computing device 30 and data storage 50 together and comprises one or more wired, wireless or combined wired/wireless networks and corresponding hardware such as hubs, switches, access points, network interfaces or other hardware commonly found in a network. Example wired and wireless networks that may be utilized include the Internet, a wide area network (WAN), a local area network (LAN), satellite, telephone, cable, a fiber-optic, cellular, ethernet, WiFi, WiMAX, Bluetooth®, any other network or connection or any combination thereof.

Computing device 30 may comprise one or more processing device(s) 32, memory 34, a network interface 36, a display device 38, an input device 40 or any other components commonly used by a computing system.

Processing device(s) 32 may comprise, for example, a processor, a microprocessor, a microcontroller (MCU), an application-specific integrated circuit (ASIC), a field-programmable gate array (FPGA), a graphics processing unit (GPU), a printed circuit board (PCB), or any other processing device.

Network interface 36 may comprise a wired or wireless communication interface that is configured to communicate with optical test strip reader 200 and data storage 50, e.g., via network 20. Example wired and wireless connections and networks that may be integrated into or utilized by network interface 36 include the Internet, a wide area network (WAN), a local area network (LAN), satellite, telephone, cable, a fiber-optic, cellular, ethernet, WiFi, WiMAX, Bluetooth®, any other network or connection or any combination thereof.

Display device 38 comprises, e.g., a screen, a monitor, a television, phone, smart device, or any other device that is configured to present data or images to a user.

Input device 40 comprises, e.g., a keyboard, mouse, touch screen, or any other physical interface that is configured to receive a user input from a user.

Data storage 50 comprises data storage devices or data storage systems that are configured for storage of large volumes of data that may be utilized by computing device 30. Example data storage devices that may be utilized by data storage 50 include hard disk drives (HDD), solid state drives (SSDs) or other storage technologies. In some embodiments, the data storage devices may be implemented using non-volatile memory (NVM) devices such as flash memory. Other types of NVM devices that can be used to implement at least a portion of the data storage devices include non-volatile random access memory (NVRAM), phase-change RAM (PC-RAM) and magnetic RAM (MRAM). These and various combinations of multiple different types of NVM devices may also be used. The particular storage devices used may be varied in other embodiments, and multiple distinct storage device types may be used within a data storage system. The term “storage device” as used herein is intended to be broadly construed, so as to encompass, for example, flash drives, solid state drives, hard disk drives, hybrid drives or other types of storage devices. Example data storage systems that may be utilized by data storage 50 include network-attached storage (NAS), storage area networks (SANs), direct-attached storage (DAS) and distributed DAS, as well as combinations of these and other storage types, including software-defined storage. Other types of data storage systems that can be used including all-flash and hybrid flash storage arrays, software-defined storage systems, cloud storage systems, object-based storage systems, and scale-out NAS clusters and associated accelerators. Combinations of multiple ones of these and other data storage systems can also be used in implementing a given data storage system in an illustrative embodiment.

### Modular Optical Test Strip Reader

With reference to FIGS. 1-5, optical test strip reader 200 comprises a chassis 210, circuitry 230, one or more instrumentation modules 250, a display 280 and one or more activatable elements 290.

Optical test strip reader 200 is an easily configurable sensor platform that may be used by users to create sensor devices that measure the concentration of various analytes. Certain sub-systems of optical test strip reader 200 may be common to more than one configuration or may be more likely to be utilized by many different applications or use cases. Those sub-systems may be integrated within a main module of the optical test strip reader 200. The main module may also be referred to herein as the Reagent Functionalization Tool (RFT) of optical test strip reader 200. For example, the RFT may comprise: chassis 210, circuitry 230 such as, e.g., a motherboard PCB, a battery charging circuit, a power management sub-system, a computer processor, electronic memory, a data warehouse controller, a removable memory socket, a communication interface controller such as USB, and a wireless communication module, battery 248, display 280, activatable elements 290 such as, e.g., pushbutton switches, a touchscreen controller for display 280, or other activatable elements, and one or more connectors 249 which are configured to serve as a data interface between circuitry 230 of the RFT and instrumentation modules 250 that are connected to the RFT.

Instrumentation modules 250 may comprise, for example, a light source module 252 comprising a light source, one or more optical sensor modules 254 comprising optical sensors or photodetectors such as, e.g., photodiodes, and other components which may be configurable or otherwise selectable by the user for assembly into optical test strip reader 200, e.g., based on the user's target use case or application. For example, one or more instrumentation modules 250 may be selected for assembly based on a target analyte to be measured, one or more properties of a sample in which the target analyte may be present, potential interferent elements or any other property of the user's target use case or application. In an example, a user's target use case may comprise a detection of copper in a sample liquid. A light source module 252 may be selected that targets a peak lambda max associated with absorption by copper ions in the presence of a corresponding reagent. One or more optical sensor modules 254 may be selected that are configured to detect light at the wavelengths emitted by selected light source module 252. In this manner, changes in the absorption of light emitted by light source module 252 by the sample before and after the mixing of the reagent with the sample may be determined and utilized to determine a concentration of copper ions in the sample.

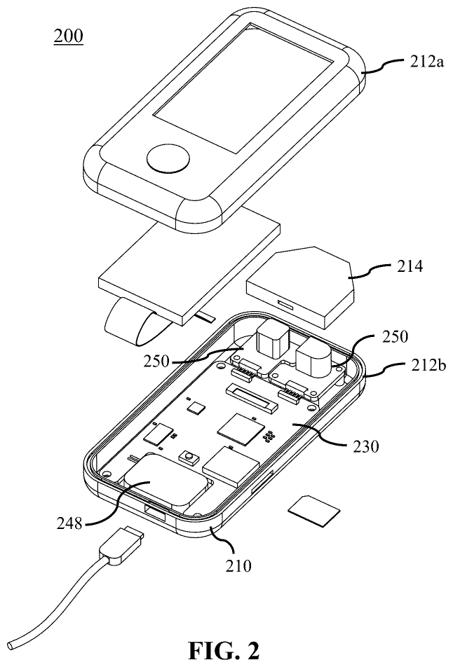

Chassis 210 comprises one or more shell components 212 and a bulkhead 214 that define a shape of chassis 210. For example, as shown in FIGS. 2-4, chassis 210 comprises shell components 212a and 212b, which may also be individually and collectively referred to herein as shell component(s) 212. Shell components 212 are configured to assembly together with bulkhead 214 such that optical test strip reader 200 may be sealed against the intrusion of environmental elements into optical test strip reader 200.

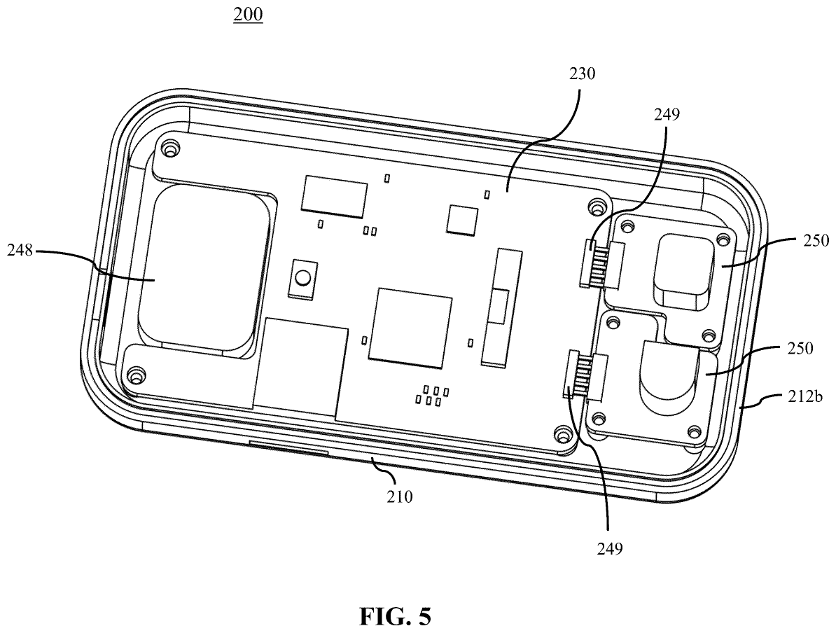

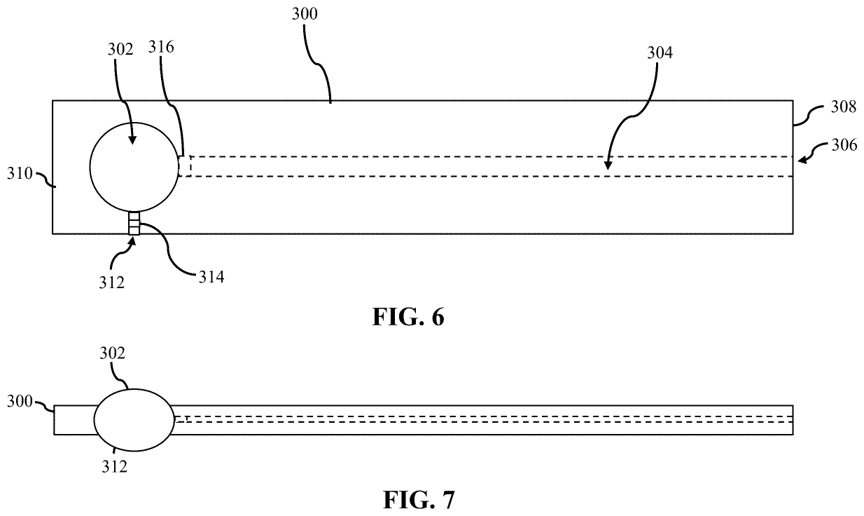

Bulkhead 214 comprises a test strip inlet 216 that is configured to receive a second end portion 310 (FIG. 6) of an optical test strip 300. In some embodiments, test strip inlet 216 may extend through a surface of bulkhead 214. While two shell components 212 and bulkhead 214 are illustrated in FIGS. 2-5, chassis 210 may alternatively comprise a larger or smaller number of shell components 212 including, e.g., a single shell component, two shell components, four shell components or any other number of shell components that may be secured together with bulkhead 214 to form chassis 210. In some embodiments, bulkhead 214 may alternatively be integrated into one of shell components 212.

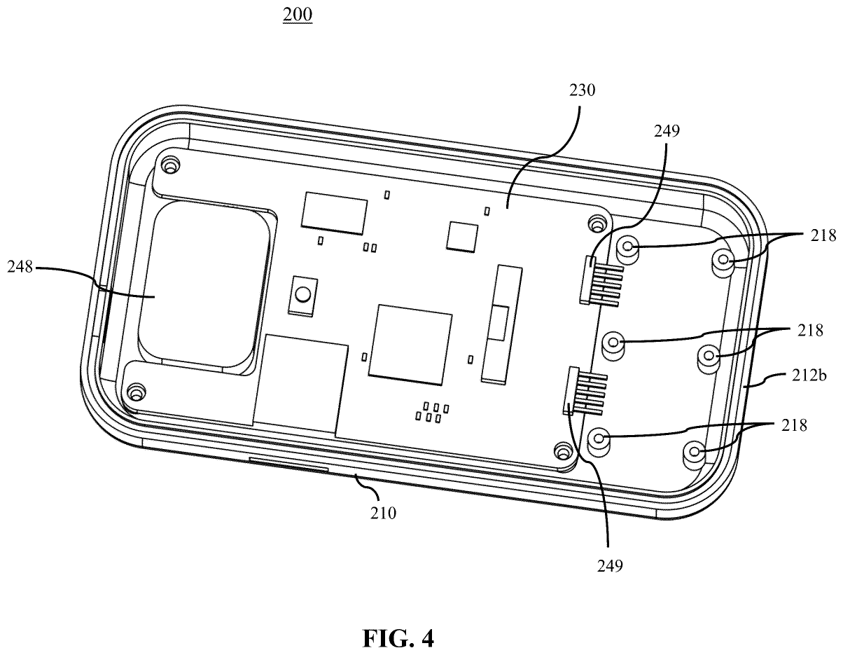

Chassis 210 may contain features that facilitate installation of specialized instrumentation modules in various positions and orientations relative to the RFT, and may contain features that facilitate the design of new modules that will be compatible with RFT integration. For example, the RFT may comprise mounting features 218 (FIG. 4) such as screw holes that are oriented in a grid pattern, and may contain excess mounting features such that combinations of modules may be inserted at various positions and orientations in order to create various instrumentation configurations within the optical test strip reader 200.

RFTs may be manufactured at scale, for example in large batches using automatic manufacturing technologies such as injection molding and pick-and-place PCB assembly robots. In this manner, the cost of an individual RFT unit may be minimized. Identical RFTs may be configured by users for creating various specialized optical test strip readers 200, with each optical test strip reader 200 utilizing an identical RFT unit. In this manner, all users of the system benefit from the value of mass production while still being able to obtain and use a specialized optical test strip reader 200 that is configured for their use case or application. In this manner, optical test strip readers 200 may be made available for a large variety of applications and uses cases at a relatively low cost with high availability and reduced production time.

In addition, sub-system designs of the RFT unit may be improved over time without impacting the various modules that may be added to the RFT to complete the optical test strip reader 200 based on the target use case or application. For example, the firmware of the RFT may be revised in order to improve data bandwidth, or correct non-obvious imperfections in the functionality of systems. The non-obvious imperfections may be discovered over the course of years by engineers, or may be reported by users of the system who have used the system for extended periods of time. Firmware updates may be distributed in order to improve functionality of optical test strip readers 200 that are in use, e.g., via a memory device such as a USB memory device, via a hardwired connection to a network or computer, via a wireless connection, via a WIFI connection, via a cellular connection, via a satellite connection or in any other manner. The hardware design of RFT units may also be improved in a similar fashion, by adopting revised designs over time. All users benefit from utilizing a common RFT unit design, because each time a new optical test strip reader 200 is created using the RFT as the platform, the most mature design is inherited. For example, optical test strip readers 200 created using the RFT may be more reliable and higher performing than optical test strip readers created from scratch for each particular use case or application.

While RFTs are described herein with respect to an optical test strip reader 200, in other embodiments, RFTs may be utilized with any other optical sensor device or other sensor device including sensor devices having a sealed sample window, sensor devices configured for insertion into an industrial process stream or other fluid flow for temporary or continuous monitoring, sensor devices configured for insertion into liquid holding tanks, mixer-settler tanks, or any other sensor device that may benefit from the use of an RFT in conjunction with configurable use-case and application specific modules. Examples of such other sensor devices that may utilize RFTs such as those described herein include sensor devices described in U.S. patent application Ser. No. 18/340,967, entitled “AUTONOMOUS MEASUREMENT SYSTEM FOR PERFORMING CHEMICAL CONCENTRATION MEASUREMENTS IN AN INDUSTRIAL PROCESS STREAM,” filed on Jun. 26, 2024, PCT Patent Application No. PCT/US23/74423, entitled “AUTONOMOUS MEASUREMENT SYSTEM FOR PERFORMING CONCENTRATION MEASUREMENTS IN A FLUID STREAM,” filed on Sep. 17, 2023 and U.S. Provisional Ser. No. 68/647,618 , entitled “MICROELECTRONIC CHEMICAL CONCENTRATION SENSOR FUNCTIONALIZATION TOOL,” filed on May 15, 2024 which are each incorporated by reference herein in their entirety.

Circuitry 230 comprises one or more printed-circuit-boards (PCBs) 232, at least one processing device 234, memory 236, an analog-to-digital converter (ADC) 238, a digital-to-analog converter (DAC) 240, a communication interface 242, a power supply 244, sensor interfaces 246, or any other type of circuitry, as well as portions or combinations of such circuitry elements.

Processing device 234 comprises, for example, a microprocessor, a microcontroller (MCU), an application-specific integrated circuit (ASIC), a field-programmable gate array (FPGA), a graphics processing unit (GPU), a programmable logic controller (PLC) or any other type of processing circuitry.

Memory 236 comprises, for example, random access memory (RAM), read-only memory (ROM), flash memory or other types of memory, in any combination. Memory 236 and other memories disclosed herein should be viewed as illustrative examples of what are more generally referred to as “processor-readable storage media” that may store executable program code of one or more software programs.

Communication interface 242 may be configured to wired or wirelessly communicate with computing device 30, data storage 40, a server, a database or any other computing device, e.g., via network 20, a wired or wireless network, mobile network, satellite network or in any other manner. The computing device 30, data storage 40, a server, a database or other computing device may be utilized to store or process data received from the optical test strip reader 200 or present the data to an end-user. In some embodiments, communication interface 242 may comprise a serial communication interface in accordance with RS-485 standards. Other example wired and wireless connections and networks that may be integrated into or utilized by communication interface 242 include the Internet, a wide area network (WAN), a local area network (LAN), satellite, telephone, cable, a fiber-optic, cellular, ethernet, WiFi, WiMAX, Bluetooth®, any other network or connection or any combination thereof.

Light source module 252 is configured to illuminate a fluid sample contained within a sample region of optical test strip 300 in order to cause the fluid sample to fluoresce, absorb or reflect light. The brightness of light source module 252 may be modulated by circuitry 230, for example, by DAC 240 which is in communication with processing device 240. In some embodiments, light source module 252 comprises an electronically modulated precision light source, for example, one or more light emitting diodes (LEDs). As an example, light source module 252 may comprise several LEDs which are multiplexed by a switching circuit of circuitry 230 to DAC 240 in some embodiments. Other types of light sources 252 may also or alternatively be utilized including, for example, lasers xenon arc lamp, phosphor-based white light sources or incandescent lamps.

Optical sensor modules 254 are disposed on circuitry 230 and positioned within chassis 210 about or adjacent to the test strip inlet 216 such that they are optically exposed to a fluid sample contained in the sample region of optical test strip 300 when optical test strip 300 is inserted into test strip inlet 216. Optical sensor modules 254 may comprise, for example, photodiodes, cameras or any other type of optical sensor in any combination. In some embodiments, optical sensor modules 254 may comprise one, two, three, four, five, six or any other number of optical sensor modules 254. In some embodiments, for example, one or more optical sensor modules 254 may be radially arranged about test strip inlet 216. In other embodiments, optical sensor modules 254 may be arranged in any other manner relative to test strip inlet 216. In some embodiments, optical sensor modules 254 may comprise one or more non-dispersive optical sensor modules 254. An instrumentation module 250 may comprise one or more optical sensor modules 254 arranged in any configuration. For example, in some embodiments, a particular instrumentation module 250 may comprise a single optical sensor module 254. In other embodiments, a particular instrumentation module 250 may comprise multiple optical sensor modules 254, e.g., 2, 3, 4, 5, 6, 7, 8, 9, 10, 11, 12 or any other number of specialized instrumentation modules 250.

### Interchangeable Bulkhead

Chassis 210 is configured to retain and precisely control the position of instrumentation modules 250. For example, chassis 210 may comprise bulkhead 214 that is attached to mounting features of the RFT. Portions of bulkhead 214 may extend to the outer surface of optical test strip reader 200, e.g., via an opening between shell components 212 when they are assembled together, and may be configured to form an air-tight seal with shell components 212.

Bulkhead 214 comprises one or more openings that are configured to receive a sample container such as a test strip or a sample tube. For example, bulkhead 214 comprises test strip inlet 216. In some embodiments, bulkhead 214 may also or alternatively comprise a sampling well that is configured for receiving a liquid sample, an opening for receiving a test tube, cuvette or other sample container, or any other opening for receiving a sample. The opening in bulkhead 214 is configured to control an alignment of features of bulkhead 214 with features of the sample container. For example, test strips may comprise a notch which may be simultaneously configured to align the test strip with test strip inlet 216 and inhibit the test strip from being inserted in orientations other than an intended orientation. In another example, a sample tube may comprise a keyway that is configured to align an optical aperture of the sample tube with instrumentation module 250 comprising an optical sensor module 254, e.g., a photodetector, of test strip reader 200.

Bulkhead 214 may comprise communication elements such as, e.g., fiber optic cables, tubes, or electrical contacts; that are configured to interact with materials contained withing the sample container.

Bulkhead 214 may be constructed of plastic such as a nylon or an epoxy that is black or opaque in order to block or absorb ambient light which would otherwise contribute to error in measurements.

Bulkhead 214 may also be specialized and modular in a similar manner to instrumentation modules 250, for example, depending on the specific instrumentation modules 250 being included in optical test strip reader 200 for a particular user and orientation of the instrumentation elements contained within modules 250, e.g., light source module 252 and optical sensor modules 254. Bulkhead 214 may be manufactured in a way that facilitates the creation of customized bulkheads 214, for example, using 3D printing or CNC machining, or the creation of a set number of bulkhead 214 configurations that correspond to a variety of instrumentation module 250 configurations. In this manner, bulkhead 214 is also specialized and may be configured for a particular use case or application in conjunction with instrumentation modules 250.

For example, a different bulkhead 214 may be selected depending on the number and type of instrumentation modules 250 to be included in optical test strip reader 200, the type of sample container, e.g., test strip, vial, cuvette, well, etc., or any other parameter associated with the particular use case or application desired by the user. By enabling the use of different bulkhead 214 configurations with both instrumentation modules 250 and the RFT, the total number of unique parts that are needed to configure optical test strip reader 200 for a particular use case or application may be reduced as compared to the need to design a test strip reader from scratch for the particular use case or application.

Multi-purpose bulkheads 214 may also be mass-produced in order to minimize the cost of an individual bulkhead 214. For example, bulkheads 214 may comprise pockets that fit more than one common-shaped instrumentation module 250 such that the most common configurations of optical test strip reader 200 are able to utilize a general-purpose bulkhead 214.

### Engineered Test Strips

With reference now to FIGS. 1, 6 and 7, optical test strips 300 are configured to draw a fluid sample into a sample region 302. For example, an optical test strip 300 may comprise a fluid pathway 304 between a fluid inlet 306 at a first end portion 308 of optical test trip 300 and sample region 302, which is disposed at a second end portion 310 of optical test strip 300. Second end portion 310 of optical test strip 300 is configured for insertion into test strip inlet 216 of optical test strip reader 200 such that sample region 302 is positioned within bulkhead 214 of optical test strip reader 200 relative to instrumentation modules 250 such as, e.g., light source module 252 and one or more optical sensor modules 254, e.g., between or adjacent to light source module 252 and one or more optical sensor modules 254.

The term activated sample refers to a reagent material that has come in contact with or has been mixed with the sample fluid intended to be measured. Optical test strip 300 is a type of sample container that acts to position the activated sample within the target region of the instrumentation modules 250. For example, optical test strip 300 may contain a sample region 302 such as, e.g., a well that is cylindrical in shape, with one or more surfaces being transparent so that radiant power from an excitation light source may illuminate the activated sample, and emissions transduced by the activated sample may pass through a transparent surface, and be communicated with one or more optical sensor modules 254 such as, e.g., photodetectors.

Optical test strip 300 may be constructed from small amounts of economical materials such as paper or plastic, allowing for optical test strips 300 to be disposable. Each measurement is performed using a clean new optical test strip 300 with the optical test strip 300 acting as a liner which isolates fluids within the test strip, keeping surfaces of the instrumentation modules 250 clean.

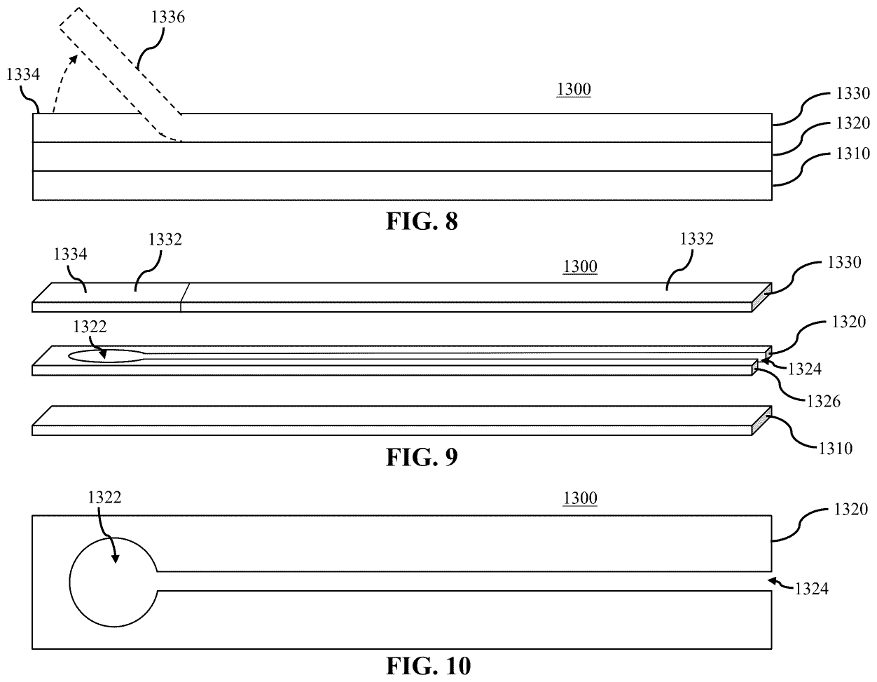

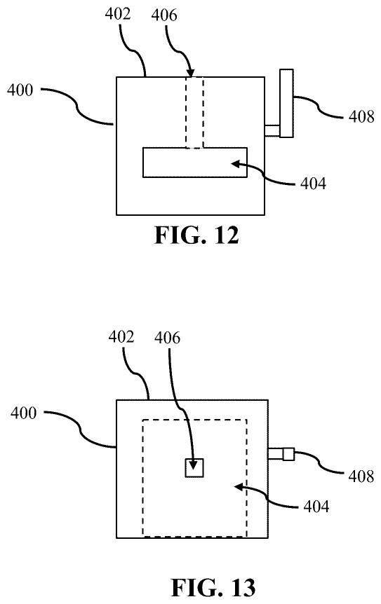

With reference to FIGS. 8-11, in an embodiment, optical test strips may be constructed from several layers of sheet or film materials which, when stacked and bonded together, form a 3D system. For example, as shown in FIGS. 8-10, an optical test strip 1300 may be formed using three layers of plastic film. The bottom layer 1310 is continuous, forming a base layer that acts to seal optical test strip 1300. The middle layer 1320 may contain 2D cutouts, for example, a circular cutout 1322 with a channel 1324 extending radially to the edge 1326 of the middle layer 1320. The top layer 1330 may be fabricated from a continuous sheet of transparent plastic film, similar to bottom layer 1310. In some embodiments, a first portion 1332 of top layer 1330 comprises a permanent adhesive and second portion 1334 of top layer 1330 comprises a tacky adhesive. The second portion 1334 overlaps the circular cutout 1322 of middle layer 1320, forming a liftable flap 1336. A reagent, for example, a powder reagent, a liquid reagent, a reagent-impregnated membrane or another type of reagent that is selected and configured for the user's target use case or application, may be inserted into circular cutout 1322 of optical test strip 1300 by lifting the flap 1336 as shown in FIG. 8, e.g., using optical test strip loader 400 (FIGS. 12 and 13).

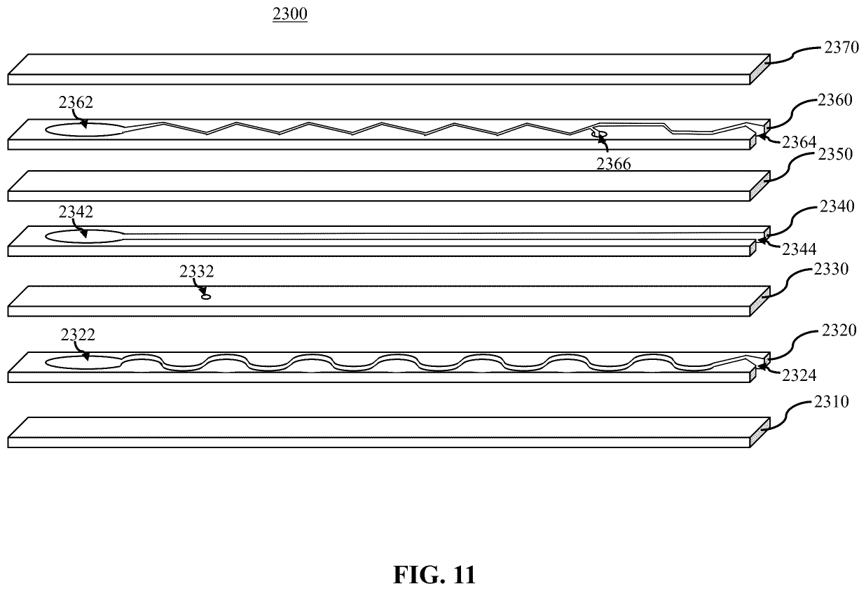

With reference to FIG. 11, an optical test strip 2300 is illustrated in another embodiment. In this embodiment, optical test strip 2300 comprises more than three layers, e.g., seven layers as shown or any other number of layers. For example, optical test strip 2300 comprises a bottom layer 2310, a first middle layer 2320, a first middle barrier layer 2330, a second middle layer 2340, a second middle barrier layer 2350, a third middle layer 2360, and a top layer 2370. Bottom layer 2310, top layer 2370 and middle barrier layers 2330 and 2350 may function in a similar manner to bottom layer 1310 and top layer 1330 as described above. Second middle layer 2340 may also function in a similar manner to middle layer 1320 as described above. In this embodiment, additional example middle layers 2320 and 2360 are provided and represent microfluidic pathways that are configured to control a flow of a sample liquid through optical test strip 2300 and also, in the case of middle layer 2360, create a mixing effect of a reagent with the sample liquid as it travels toward the sample region.

As seen in FIG. 11, middle layers 2320, 2340 and 2360 each comprise corresponding sample region 2322, 2342 and 2362 and channels 2324, 2344 and 2364. In this embodiment, middle layer 2340 comprises a channel 2344 similar to channel 1324. Channel 2324 provides a sinuous microfluidic pathway to sample region 2322 while channel 2364 provides a jagged microfluidic pathway to sample region 2362. Microfluidic pathways having sinuous or jagged channels may facility the movement of the sample through the optical test strip 2300 to sample regions 2322 and 2362.

In an embodiment, middle layer 2360 further comprises a reagent reservoir 2366 in fluid communication with channel 2364. Reagent reservoir 2366 is configured to dispense reagent at a rate corresponding to the microfluidic properties of channel 2364 as the liquid sample flows through channel 2364 toward sample region 2362, thereby mixing the reagent with the liquid sample.

In an embodiment, middle barrier layer 2330 may comprise a liquid through hole 2332 or via that is configured to transfer liquid from one of middle layers 2320 and 2340 to the other of middle layers 2320 and 2340. While through hole 2332 is provided as an example, it is understood that any middle barrier layer may comprise one or more through holes 2332 that may be utilized to transfer liquid samples or other liquids from one middle layer to another, e.g., to enable the inclusion of multiple reagents or samples on a single middle layer. For example, in some embodiments, a middle layer, e.g., middle layer 2340, situated in the middle of optical test strip 2300 may comprise multiple sample regions 2342 where one or more other middle layers supply the liquid sample and/or the reagent to one of the sample regions 2342, thereby enabling the instrumentation modules 250 to be focused on sample regions in one layer rather than multiple layers. In some embodiments, for example, only the topmost middle layer, e.g., middle layer 2360 in FIG. 11, may comprise sample regions 2362 since middle layer 2360 is the closes to the top layer 2370 and may enable optical measurements or excitation by instrumentation modules 250 with the smallest distance between the instrumentation modules 250 and the corresponding sample region.

While optical test strips 1300 and 2300 are described herein with bottom, middle and top layers, it is understood that such layers and other optional layers may be formed in any order or configuration to form optical test strips 1300 and 2300, e.g., depending on the particular use case or application for which the user is customizing the optical test strips 1300 and 2300.

Optical test strips 300, 1300 and 2300 may contain standardized features, allowing interchangeable optical test strips to be inserted into a common bulkhead 214, or to interface with common instrumentation modules 250.

Sample regions 302, 1322, 2322, 2342 and 2362 comprise a micro-fluidic cavity that is configured to hold a known volume of a fluid sample. In some embodiments, sample regions 302, 1322, 2322, 2342 and 2362 may be impregnated with reagents that are configured to interact with a target fluid sample. For example, the reagents may be applied to sample regions 302, 1322, 2322, 2342 and 2362 as coatings, powders, or in any other manner, e.g., during assembly or construction of optical test strips 300, 1300 and 2300. In some embodiments, membranes or other materials may be impregnated with the reagents and positioned within sample regions 302, 1322, 2322, 2342 and 2362. The reagents may be configured or specially tailored to interact with a particular target analyte of interest. For example, the reagents may be configured to cause a change in color of the fluid sample in the presence of the target analyte in the fluid sample. In the absence of the target analyte in the fluid sample, no color change may occur. The amount of color change may be proportional to the concentration or another chemical property of the target analyte in the fluid sample.

In some embodiments, the reagents may be configured to induce a fluorescence in the fluid sample in the presence of the target analyte. For example, the reagents may interact with the target analyte to cause the fluorescence at a particular wavelength or series of wavelengths. In some embodiments, the fluorescence may occur due to excitation of the reagent, the target analyte or both by light source module 252.

In some embodiments, the reagents may be configured to affect absorption characteristics of the target analyte in the fluid sample. For example, the reagents may shift the wavelength of light that is absorbed by the target analyte, may cause an increase or decrease in the absorption of a particular wavelength of light by the target analyte or may affect absorption by the target analyte in any other manner.

In use, first end 308 of optical test strip 300 is positioned in a fluid being tested such that fluid inlet 306 is in contact with the fluid. The fluid is drawn into sample region 302 via fluid inlet 306 and fluid pathway 305, e.g., by capillary action or another fluid mechanism, such that a predetermine volume of fluid (the fluid sample) is disposed in sample region 302. The fluid sample is mixed with the reagent disposed within sample region 302, if any. In some embodiments, for example, a user may shake optical test strip 300 to mix the fluid sample with the reagent. In other embodiments, the capillary action pulling the fluid into the sample region 302 may automatically mix the fluid sample with the reagent. The optical test strip 300 has now been prepared for measurement.

When second end portion 310 of optical test strip 300 is inserted into optical test strip reader 200, optical sensor modules 254 alone or in conjunction with light source module 252 may be utilized to detect characteristics of the fluid sample contained within sample region 302 of optical test strip 300. For example, optical sensor modules 254 may be configured to detect color, changes in color, fluorescence at one or more wavelengths, absorption or any other optical characteristic of a fluid sample contained in sample region 302. In some embodiments, light source module 252 and optical sensor modules 254 may be pre-configured to detect certain characteristics of the fluid sample, e.g., based on the target analyte being measured, the reagent being utilized, characteristics of the fluid or any other relevant information.

While illustrated as a flat rectangular optical test strip 300 in FIGS. 6 and 7, or flat layered optical test strips 1300 and 2300 in FIGS. 8-11, in other embodiments, optical test strips 300, 1300 or 2300 may comprise any other shape including, e.g., cylindrical, cuboid or any other shape. For example, a cuboid optical test strip version of optical test strips 1300 or 2300 may comprise layers that are stacked in more than one direction, e.g., bottom to top and also side-to-side.

Sample regions 302, 1322, 2322, 2342 and 2362 may comprise three dimensional volumes that may be ovoid, spherical, cuboid or have any other shape. As shown in FIGS. 6 and 7, sample region 302 may extend out of a body of optical test strip 300. In other embodiments, sample region 302 may be fully contained within the body of optical test strip 300 where, for example, the body of optical test strip 300 may comprise a transparent or other material that enables optical detection of the contents of sample region 302 at various target wavelengths and colors of light.

The optical test strips described herein may contain intricate engineered features, which act to interface the activated sample with one or more instrumentation systems. For example, the described optical test strips may comprise some or all of printed circuit layers, microfluidic circuits, porous materials, hydrophilic materials, hydrophobic materials, electrical contacts, wettable conductive materials such as woven carbon nanotubes, electrophoresis gel, layers containing pockets of liquid which release upon insertion of the test strip, fiber optic cables, optical windows, and mirrored surfaces.

Test strips may comprise one or more stiffener layers, which may act as a substrate onto which thinner layers are deposited. Stiffener layers may be stacked in order to protect delicate features disposed between.

Layers of the optical test strips may comprise channels of filter material. When the optical test strip is dipped into a liquid sample, for example river water, the filter material may act to wick fluid into the sample region of the optical test strip by capillary action. While fluid is being wicked, particulate contaminants may be trapped by the filter material, allowing only filtered fluid to enter the target region and be mixed with reagent. In this manner, for example, grains of organic debris in the river water will be captured by the filters and will be inhibited from masking light intended to be transmitted to the instrumentation modules 250 from the sample region.

Optical test strips 300, 1300 and 2300 may contain electrical conductors allowing electrical instrumentation to communicate with the sample. For example, microscopic platinum contacts disposed within the target region of the optical test strips 300, 1300 and 2300 may form an electrical connection with an instrumentation module within the optical test strip reader 200 containing an ADC and a precision current source. In this manner, electrical conductivity of the sample fluid may be measured by the test strip reader.

### User Configurable Optical Test Strips

In some embodiments, optical test strips 300, 1300 and 2300 may be provided without any reagent and may be configured to enable an end-user to load a reagent of choice into one or more of sample regions 302, 1322, 2322, 2342 and 2362. As an example, optical test strip loader 400 may be configured to load a reagent of choice into sample region 302, e.g., using the tacky flap as described above or in another manner.

For example, multi-purpose optical test strips may comprise features that enable users to install reagent into the optical test strips. For example, multi-purpose optical test strips may be utilized during scientific research, by preparing various reagent formulations in a laboratory, then assembling various groups of corresponding optical test strips. Many multi-purpose optical test strip designs may be compiled into a library, allowing user to select an appropriate optical test strip for their reagent. Multi-purpose optical test strips may be utilized during characterization testing of reagents, in order to optimize the optical test strip design and optimize the optical test strip reader 200's firmware.

Other multi-purpose optical test strips may be optimized for mass production by automatic equipment. For example, mass-produced optical test strips may utilize extremely small quantities of reagent, or may be configured for robotic assembly at extremely high speed. Test strips may be fabricated in sheets containing hundreds of test strips, which are laminated together as a single sheet, then diced into strips after reagent is installed.

In an embodiment, optical test strips 300 may comprise a mechanism that enables loading of a reagent into sample region 302. For example, sample region 302 may be configured as a clamshell that is openable by optical test strip loader 400 but otherwise remains fluidly sealed, in a similar manner to the tacky flap 1336 of FIG. 8. As an example, the second end 310 of optical test strip 300 may be inserted into optical test strip loader 400 and the clamshell or flap 1336 may be opened by a mechanical action of optical test strip loader 400. In an example, the act of inserting the second end 310 of optical test strip 300 into optical test strip loader 400 may cause the mechanical action, e.g., by pressing against a lever or other actuation mechanism which causes optical test strip loader 400 to open the clamshell. In other embodiments, a user actuation of a button, lever or other mechanism may cause the opening of the clamshell after optical test strip 300 is inserted into optical test strip loader 400. In some embodiments, optical test strip loader 400 may comprise electrical components, actuators, buttons, etc. that are configured to control the opening or access to sample region 302 of optical test strip 300 for the impregnation of sample region 302 with a reagent.

In another embodiment, a separate pathway 312 may be opened by optical test strip loader 400 to impregnate sample region 302 with the reagent, e.g., by removing a plug, opening a one-way valve or in another manner. As an example, optical test strip 300 may comprise a pathway 312 for loading reagent into sample region 302 that comprises a plug or one-way valve 314 that is actuatable by optical test strip loader 400. In some embodiments, fluid pathway 304 may also comprise a one-way valve 316 to ensure that the reagent does not exit sample region 302 via fluid pathway 304.

In another embodiment, optical test strips may be in the form of a cylindrical sample vessel. Bulkheads 214 may contain sockets that are configured to receive industry-standard containers, so that sensor devices can be created which are compatible with widely available sample containers. For example, the vessel may be a glass vial with a screw-on cap, or an injection-molded microcentrifuge tube, or a segment of fiberoptic cable containing a cylindrical bore at one end. The cylindrical sample container may be coated with the reagent, may contain a piece of porous material that is impregnated with the reagent or may be configured to deploy the reagent in any other manner.

### Optical Test Strip Loader

With reference to FIGS. 12 and 13, optical test strip loader 400 comprises a housing 402, a test strip inlet 402, a reagent inlet 404 and an actuator 406. Test strip inlet 402 extends into housing 400 such that the sample region 302 of an optical test strip 300 inserted into test strip inlet 402 is positioned adjacent reagent inlet 404. In some embodiments, reagent inlet 404 may be configured to drop a reagent into sample region 302 when sample region is opened by actuator 406, e.g., where sample region 302 is a clamshell. In other embodiments, reagent inlet 404 may be configured in fluid communication with pathway 312 when an optical test strip 300 is inserted into test strip inlet 402 such that the reagent may be delivered to sample region 302 via reagent inlet 404 and pathway 312.

Users may prepare optical test strips using a test strip filling tool. For example, when empty test strips are inserted into the tool, wedge-shaped dyes of the tool may act to cleave or lift the flap of test strips. When fully inserted, the target region of the fillable test strip is positioned in line with a funnel. Reagent may be deposited into the funnel or into the test strip using a metering tool such as a micropipette.

Actuator 408 may comprise a mechanical or electrical actuator that is configured to deliver a predetermined amount of reagent to sample region 302. For example, in the case of a mechanical actuator 408, one pull of actuator 408 may cause a predetermined amount of reagent to enter sample region 302. In an embodiment, actuation of actuator 408 may cause the clamshell to open and the predetermined amount of reagent to enter sample region 302 while a release of the actuator 408 may cause the clamshell to close. In other embodiments, the actuation of actuator 408 may cause a mechanical or electrical component to push or otherwise supply a predetermined amount of reagent to sample region 302, e.g., via pathway 312.

A first example optical test strip is disclosed. The first example optical test strip comprises a bottom layer; a middle layer comprising a microfluidic well and a microfluidic channel extending to an end surface of the middle layer; a top layer, the middle layer being disposed between the bottom and top layers such that the microfluidic well defines a sample region and the microfluidic channel defines a fluid pathway from a sampling end of the optical test strip to the microfluidic well; and a reagent disposed in the microfluidic well and configured to interact with a liquid sample drawn into the microfluidic well via the fluid pathway.

A second example optical test strip is disclosed. The second example optical test strip comprises a bottom layer; a middle layer comprising a microfluidic well and a microfluidic channel extending to an end surface of the middle layer; a top layer, the middle layer being disposed between the bottom and top layers such that the microfluidic well defines a sample region and the microfluidic channel defines a fluid pathway from a sampling end of the optical test strip to the microfluidic well; and at least one loading feature that is configured to enable a user to load a reagent into the microfluidic well.

A third example optical test strip is disclosed. The third example optical test strip comprises a bottom layer; a top layer; a plurality of middle layers, a given middle layer of the plurality of middle layers comprising a microfluidic well and a microfluidic channel extending from the microfluidic well and having one of a sinuous and a jagged profile, the microfluidic channel being configured to transport a fluid sample to the microfluidic well, the plurality of middle layers being disposed between the bottom and top layers; and a reagent disposed in one of the plurality of middle layers and being configured to mix with the fluid sample in at least one of the microfluidic well and the microfluidic channel.

An example optical test strip loader is disclosed. The example optical test loader comprises a reagent fluid inlet; an optical test strip inlet that is configured to receive an optical test strip therein; and an actuation mechanism that is configured to engage with the optical test strip to open a fluid pathway between the reagent fluid inlet and a microfluidic well of the optical test strip.

FIGS. 1 through 13 are conceptual illustrations allowing for an explanation of the disclosed embodiments of the invention. Notably, the figures and examples above are not meant to limit the scope of the invention to a single embodiment, as other embodiments are possible by way of interchange of some or all of the described or illustrated elements. Moreover, where certain elements of the disclosed embodiments can be partially or fully implemented using known components, only those portions of such known components that are necessary for an understanding of the disclosed embodiments are described, and detailed descriptions of other portions of such known components are omitted so as not to obscure the disclosed embodiments. In the present specification, an embodiment showing a singular component should not necessarily be limited to other embodiments including a plurality of the same component, and vice-versa, unless explicitly stated otherwise herein. Moreover, terms in the specification or claims are not intended to be ascribed an uncommon or special meaning unless explicitly set forth as such. Further, the disclosed embodiments encompass present and future known equivalents to the known components referred to herein by way of illustration.

It should be understood that the various aspects of the embodiments could be implemented in hardware, firmware, software, or combinations thereof. In such embodiments, the various components and/or steps would be implemented in hardware, firmware, and/or software to perform the functions of the disclosed embodiments. That is, the same piece or different pieces of hardware, firmware, or module of software could perform one or more of the illustrated blocks (e.g., components or steps). In software implementations, computer software (e.g., programs or other instructions) and/or data is stored on a machine-readable medium as part of a computer program product and is loaded into a computer system or other device or machine via a removable storage drive, hard drive, or communications interface. Computer programs (also called computer control logic or computer-readable program code) are stored in a main and/or secondary memory, and executed by one or more processors (controllers, or the like) to cause the one or more processors to perform the functions of the invention as described herein. In this document, the terms “machine readable medium,” “computer-readable medium,” “computer program medium,” and “computer usable medium” are used to generally refer to media such as a random access memory (RAM); a read only memory (ROM); a removable storage unit (e.g., a magnetic or optical disc, flash memory device, or the like); a hard disk; or the like.

The foregoing description will so fully reveal the general nature of the disclosed embodiments that others can, by applying knowledge within the skill of the relevant art(s) (including the contents of the documents cited and incorporated by reference herein), readily modify and/or adapt for various applications such specific embodiments, without undue experimentation, without departing from the general concept of the disclosed embodiments. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

What is claimed is:

1. An optical test strip reader assembly comprising:
a chassis;
a reagent functionalization tool comprising a plurality of standard components installed in the chassis;
a plurality of instrumentation modules that are each configured for assembly with the reagent functionalization tool in the chassis, each instrumentation module being selectable for assembly with the reagent functionalization tool in the chassis based on at least one parameter that corresponds to a target application for the optical test strip reader; and
an optical test strip inlet that is configured to receive a portion of an optical test strip therein and to align a sample region of the optical test strip within the chassis at a location corresponding to a given instrumentation module of the plurality of instrumentation modules when the given instrumentation module is assembled within the chassis.

2. The optical test strip reader assembly of claim 1, wherein the at least one parameter is obtained from a computing device of a user.

3. The optical test strip reader assembly of claim 1, wherein the plurality of instrumentation modules comprises a light source module, the light source module comprising a light source.

4. The optical test strip reader assembly of claim 1, wherein the plurality of instrumentation modules comprises an optical sensor module, the optical sensor module comprising a photodetector.

5. The optical test strip reader assembly of claim 1, wherein the optical test strip reader assembly further comprises a plurality of bulkheads, each bulkhead comprising a corresponding optical test strip inlet.

6. The optical test strip reader assembly of claim 1, wherein each instrumentation module comprises an electrical contact that is configured for electrical attachment to the reagent functionalization tool when the instrumentation module is assembled with the reagent functionalization tool in the chassis.

7. The optical test strip reader assembly of claim 1, wherein the chassis comprises a plurality of mounting features, each instrumentation module being configured to attach to at least one of the mounting features when that instrumentation module is assembled with the reagent functionalization tool in the chassis.

8. The optical test strip reader assembly of claim 7, wherein at least one of the instrumentation modules is configured to attach to the at least one of the mounting features in a different orientation than at least another of the instrumentation modules.

9. A method of manufacture of an optical test strip reader, the method comprising:
obtaining at least one parameter corresponding to a target application;
identifying an instrumentation module to include in the optical test strip reader from a plurality of available instrumentation modules based on the obtained at least one parameter; and
assembling the optical test strip reader by:
obtaining a chassis;
installing a reagent functionalization tool comprising a plurality of standard components in the chassis;
installing the identified instrumentation module in the chassis, the installation of the identified instrumentation module electrically connecting the identified instrumentation module with the reagent functionalization tool;
installing a bulkhead in the chassis, the bulkhead comprising an optical test strip inlet that is configured to align a sample region of an optical test strip inserted therein with the installed instrumentation module; and
sealing the chassis.

10. The method of claim 9, further comprising obtaining the at least one parameter from a computing device of a user.

11. The method of claim 9, wherein the plurality of available instrumentation modules comprises a light source module, the light source module comprising a light source.

12. The method of claim 9, wherein the plurality of available instrumentation modules comprises an optical sensor module, the optical sensor module comprising a photodetector.

13. The method of claim 9, wherein installing the identified instrumentation module in the chassis comprises mounting the identified instrumentation module to at least one mounting feature of the chassis in an orientation corresponding to the identified instrumentation module.

14. The method of claim 13, wherein at least one of the available instrumentation modules is configured to mount to the at least one mounting feature in a different orientation than the identified instrumentation module.

15. An optical test strip reader comprising:
a chassis;
a reagent functionalization tool comprising a plurality of standard components installed in the chassis;
a first instrumentation module installed in the chassis, the first instrumentation module having been selected from a plurality of available instrument modules based on at least one parameter corresponding to a target application;
a second instrumentation module installed in the chassis, the second instrumentation module having been selected from the plurality of available instrument modules based on the at least one parameter corresponding to the target application;
a bulkhead installed in the chassis, the bulkhead comprising an optical test strip inlet that is configured to align a sample region of an optical test strip inserted therein with the first and second instrumentation modules.

16. The optical test strip reader of claim 14, wherein the at least one parameter is obtained from a computing device of a user.

17. The optical test strip reader of claim 14, wherein the first and second instrumentation modules are installed in the chassis in electrical communication with the reagent functionalization tool.

18. The optical test strip reader of claim 14, wherein the first and second instrumentation modules are installed in the chassis via attachment to corresponding mounting features of the chassis.

19. The optical test strip reader of claim 14, wherein the first instrumentation module comprises a light source module, the light source module comprising a light source that is configured to emit light at a wavelength determined based on the at least one parameter.

20. The optical test strip reader of claim 14, wherein the second instrumentation module comprises an optical sensor module, the optical sensor module comprising a photodetector that is configured to measure light at a wavelength determined based on the at least one parameter.
